(19) United States
(12) Patent Application Publication
(10) Pub. No.: US 2026/0338898 A1
(43) Pub. Date: Oct. 29, 2026

(54) IMAGE RECONSTRUCTION IN MAGNETIC RESONANCE IMAGING DEPENDING ON DATA CONSISTENCY

(71) Applicant: Siemens Healthineers AG, Forchheim (DE)

(72) Inventors: Daniel Polak, Blankenbach (DE); Stephen Farman Cauley, Winchester, MA (US); Bryan Clifford, Belmont, MA (US); Daniel Nicolas Splitthoff, Uttenreuth (DE)

(21) Appl. No.: 19/656,235

(22) Filed: Apr. 23, 2026

(30) Foreign Application Priority Data
Apr. 25, 2025 (EP) 25172648

(51) Int. Cl. G01R 33/56; G06T 3/40; G06T 5/50; G06T 5/60; G06T 7/00; G06T 7/207; G06V 10/764; G06V 10/82; G06V 40/20

(52) U.S. Cl. CPC G01R 33/5608; G06T 3/40; G06T 5/50; G06T 5/60; G06T 7/0016; G06T 7/207; G06V 10/764; G06V 40/20; G06T 2207/20016; G06T 2207/20081; G06T 2207/20084; G06T 2207/30016; G06T 2207/30168; G06V 10/82; G06V 2201/031

(57) ABSTRACT

For image reconstruction in magnetic resonance imaging, MRI data representing an object is received. A first preliminary image is reconstructed based on the MRI data. A first data consistency measure of the first preliminary image with respect to the MRI data is computed. A final image is reconstructed based on the MRI data, wherein, depending on the first data consistency measure, a motion of the object during an acquisition of the MRI data is either compensated in the final image based on predetermined motion data, which quantifies the motion of the object during the acquisition of the MRI data, or is not compensated in the final image. Therein, a predefined image quality of the first preliminary image is less than the image quality of the final image.

### CROSS REFERENCE TO RELATED APPLICATIONS

This application claims the benefit of EP25172648.5 filed on Apr. 25, 2025, which is hereby incorporated by reference in its entirety.

### FIELD

Embodiments are related to a computer-implemented method for image reconstruction, for example retrospective image reconstruction, in magnetic resonance imaging (MRI).

### BACKGROUND

Patient motion during the acquisition of MRI data may lead to significant artifacts or distortions in the reconstructed images. Motion correction techniques to reduce artifacts in MRI images are known. For clinical applications, it is crucial that these methods work with high reliability, effectively mitigating artifacts when motion is present, while preserving image quality when the patient remains still. Achieving a high level of robustness is challenging due to technical limitations, including errors in motion tracking. These inaccuracies may sometimes lead to degradations in the image quality of the images. In some instances, images corrected for motion may even appear worse than uncorrected images, for example with subtle ringing or more pronounced motion artifacts emerging as a result of the correction techniques.

In general, motion correction techniques for MRI may be classified as prospective or retrospective motion correction techniques. In prospective motion correction, adjustments are made in real time to manage motion artifacts as they occur. This approach has a limited flexibility due to the need for immediate response but may include real-time filtering techniques such as the Kalman filter or threshold-based field-of-view updating.

Retrospective techniques allow for additional processing time, facilitating a comprehensive analysis of motion data and images. For example, motion trajectory filtering approaches involve smoothing or adjusting the estimated motion trajectory before applying any corrections.

In retrospective approaches using automated correction decision-making, an algorithm evaluates whether to display the corrected or uncorrected image to the user based on the quality. Both direct, image-based methods and indirect, trajectory-based methods may be used.

For image-based quality assessment, techniques based on image entropy or using deep learning networks have been developed to automatically evaluate and select the highest-quality image. However, reliably fine-tuning of these metrics to consistently distinguish image quality may be challenging, especially across varying sequence protocols, contrast settings, and noise conditions. Additionally, these approaches require generating both the corrected and uncorrected images, which is computationally intensive. If the corrected image ultimately proves inferior, this process results in unnecessary reconstruction time. Alternatively, decisions may be made by analyzing the estimated motion trajectory. Metrics such as maximum translation and/or rotation, motion standard deviation, or other statistical measures may be applied. While these methods avoid the need for explicit image reconstruction, they are typically less reliable, often requiring a balance between sensitivity and specificity to optimize the results.

The publication D. Polak et al.: “Scout Accelerated Motion Estimation and Reduction (SAMER)”, Magn. Reson. Med., 2022, 87(1), 163-178 describes a technique denoted as SAMER, which utilizes a single short, low-resolution scout scan and sequence reordering to independently determine motion states by minimizing the data-consistency error in a SENSE plus motion forward model.

The publication D. Polak et al.: “Motion guidance lines for robust data-consistency based retrospective motion correction in 2D and 3D MRI”, Magn. Reson. Med., 2023, 89(5), 1777-1790 proposes a motion correction technique based on repeating k-space guidance lines for improving motion correction in cartesian 2D and 3D brain MRI.

The publication L. Cordero-Grande et al.: “Sensitivity Encoding for Aligned Multishot Magnetic Resonance Reconstruction”, IEEE Transactions on Computational Imaging, 2016, vol. 2, no. 3 describes a framework for the reconstruction of magnetic resonance images in the presence of rigid motion.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide an improved concept for retrospective motion compensation, also denoted as motion correction, in MRI, which features low computational costs and a high reliability.

Embodiments use a data consistency of a reconstructed preliminary image, which has a reduced image quality, with respect to the underlying MRI data as a criterion for deciding whether the final image is reconstructed with or without motion compensation.

According to a first aspect, a computer-implemented method for image reconstruction in magnetic resonance imaging, MRI, is provided. Therein, MRI data, for example measured MRI data, representing an object, for example an imaged object, is received. A first preliminary image is reconstructed based on the MRI data. A first data consistency measure of the first preliminary image with respect to the MRI data is computed. A final image is reconstructed based on the MRI data, wherein a predefined image quality of the first preliminary image is less than the image quality of the final image. Furthermore, depending on the first data consistency measure, a motion of the object during an acquisition of the MRI data is either compensated in the final image, for example when reconstructing the final image, based on predetermined motion data, which quantifies the motion of the object during the acquisition of the MRI data, or the motion of the object is not compensated in the final image.

Unless stated otherwise, all steps of the computer-implemented method may be performed by a data processing system, which includes at least one data processing device. For example, the at least one data processing device is configured to perform the steps of the computer-implemented method. For this purpose, the at least one data processing device may for example store a computer program including instructions which, when executed by the at least one data processing device, cause the at least one data processing device to execute the computer-implemented method. The expressions “data processing system” and “at least one data processing device” may be used interchangeably, here and in the following. This holds also for respective expressions derived therefrom.

In case the at least one data processing device includes two or more data processing devices, certain steps carried out by the at least one data processing device may also be understood such that different data processing devices carry out different steps or different parts of a step. For example, it is not required that each data processing device carries out the steps completely. In other words, carrying out the steps may be distributed amongst the two or more data processing devices.

From each implementation of the computer-implemented method, a respective implementation of a method for image reconstruction in MRI, which is not purely computer-implemented, is obtained by including respective steps of generating the MRI data, for example by an MRI device.

Receiving data or information, for example the MRI data, may, for example, involve receiving the data, for example by the data processing system from a sending entity, or reading the data from a data storage or receiving a data stream containing the data, or extracting the data from the data stream, and so on. For example, wired or wireless data transmission may be used for this purpose. For example, the data transmission may take place between a hardware and/or software interface of the sending entity and a hardware and/or software interface of the data processing system.

A reconstructed image may also be denoted as MRI image. A reconstructed image may also be denoted as image reconstruction. A reconstructed image may be a two-dimensional or a three-dimensional image, the latter also being denoted as reconstructed volume. A reconstructed image may also be a two-dimensional slice or layer of a three-dimensional reconstructed image. For example, the first preliminary image and the final image may also be denoted as first preliminary MRI image and final MRI image, respectively.

The MRI data are, for example, MRI data in k-space or in a hybrid image-k-space. The MRI data may include different datasets, which correspond for example to different data acquisition shots of the acquisition of the MRI data and/or different k-space regions. The different datasets of the MRI data may also include datasets corresponding to preliminary scans or navigation scans or scout scans, such as the SAMER scout scan.

Consequently, it is possible that the MRI data are not used completely for reconstructing the final image and/or for reconstructing the first preliminary image and so forth. For example, in some embodiments, only a part of the MRI data is used to reconstruct the first preliminary image. In this case, the first data consistency measure is computed for the first preliminary image with respect to the part of the MRI data, which has been used for reconstructing the first preliminary image.

Considering a reconstructed MRI image, it may be transferred back into k-space by applying the corresponding forward operator of the acquisition to the reconstructed MRI image. A data consistency measure may for example be a measure, which quantifies a deviation between the k-space data resulting from the back transferred MRI image and the original k-space data, which has been used to reconstruct the MRI image. The data consistency measure may therefore be of the form of a data consistency error ∥E x−y∥, wherein x denotes the reconstructed MRI image E denotes a predefined forward operator and y denotes the original k-space data. The forward operator may, for example, include a Fourier transform, respective coil sensitivity maps in case of parallel imaging and a projection to the sampled k-space points in case of k-space subsampling. The forward operator may also include or depend on the motion data, for example.

The predefined image quality may for example be an image size or a resolution, for example a spatial resolution, or another image quality, whose reduction leads to a reduced computational effort for reconstructing the respective image. Consequently, the computational resources required for reconstructing the first preliminary image are less than the computational resources required for reconstructing the final image in case all other relevant parameters would be identical.

According to an embodiment, it is decided depending on the first data consistency measure whether the motion of the object is compensated using the motion data when reconstructing the final image or not. For example, the motion of the object is compensated when reconstructing the final image only if it is determined based on the first data consistency measure that a sufficient benefit may be expected due to the motion compensation. In other words, motion compensation is avoided if the benefit is expected to be low or if the motion compensation may even lead to a worse result than achievable without motion compensation. Thus, the computational effort is, on average, significantly reduced without risking significant artifacts due to object motion during the acquisition.

Another advantage is that explicit image-based metrics, such as image entropy, which are often challenging to tune and may not consistently yield reliable results, are not required for the computer-implemented method.

For reconstructing the first preliminary image and final image depending on the MRI data, known methods for MRI reconstruction may be used. For example, if motion compensation depending on the motion data shall be included, known techniques for retrospective motion correction may be used.

Also the motion data may be generated using known techniques including, for example, navigator scans, scout scans, camera-based motion tracking, optimization methods and so forth.

For example, the motion data may include or consists of one, two, or three translation parameters as a function of time during the acquisition of the MRI data and/or one, two, or three rotation parameters as a function of time during the acquisition of the MRI data. In other words, the motion of the object is assumed to be a rigid motion. Alternatively, whole motion vector fields may be used, which also allows for considering non-rigid motion.

According to certain embodiments, the image quality is a spatial resolution. Consequently, the spatial resolution of the first preliminary image is less than the spatial resolution of the final image. In other words, the number of k-space samples used or considered to reconstruct the first preliminary image is less than the number of k-space samples used or considered to reconstruct the final image.

Reducing the number of k-space samples for reconstructing the first preliminary image compared to reconstructing the first preliminary image significantly reduces the computational effort for reconstructing the first preliminary image. On the other hand, object motion typically affects certain regions of the k-space, for example regions close to the k-space center, much more than other regions, for example region further away from the k-space center. Thus, by selecting the k-space samples used for reconstructing the first preliminary image accordingly, the influence of the motion of the object on the first preliminary image and, consequently, the first data consistency measure, is not significantly different compared to a case where all k-space samples would be used. The first preliminary image is therefore still a suitable measure for deciding whether the final image should be reconstructed with or without motion compensation.

According to certain embodiments, the motion of the object during the acquisition of the MRI data is compensated in the first preliminary image, for example when reconstructing the first preliminary image, based on the motion data.

In this way it is achieved that the influence of the motion of the object on the first preliminary image is reduced or completely removed. The first data consistency measure may therefore serve as a reference measure for what may be expected from the motion compensation for the final image.

According to certain embodiments, a second preliminary image is reconstructed based on the MRI data, wherein the motion of the object is not compensated in the second preliminary image, for example when reconstructing the second preliminary image, and the image quality of the second preliminary image is less than the image quality of the final image. A second data consistency measure of the second preliminary image with respect to the MRI data is computed. A data consistency deviation between the first data consistency measure and the second data consistency measure is computed. The final image is reconstructed based on the MRI data, wherein, depending on the data consistency deviation, the motion of the object is either compensated based on the motion data in the final image or is not compensated in the final image.

According to such embodiments, it is decided depending on the data consistency deviation whether the motion of the object is compensated using the motion data when reconstructing the final image or not.

For example, the data consistency deviation may be a difference or an absolute value of the difference between the first data consistency measure and the second data consistency measure.

For example, the image quality of the second preliminary image may be equal to the image quality of the first preliminary image. For example, the first preliminary image and the second preliminary image may be reconstructed based on the same part of the MRI data.

The second data consistency measure may serve as a reference measure for what may be expected for the final image if no motion compensation is carried out. The data consistency deviation may therefore be considered as a measure of the expected benefit of the motion compensation when reconstructing the final image.

For example, it may be provided that the motion of the object is compensated based on the motion data in the final image if the data consistency deviation is greater than a predefined threshold value and the motion of the object is not compensated in the final image otherwise.

According to certain embodiments, the first data consistency measure depends on or is given by ||E_{θ}x−y||, wherein x denotes the first preliminary image, θ denotes the motion data, E_{θ}denotes a predefined forward operator, which depends on the motion data, and y denotes the part of the MRI data, which is used for generating the first preliminary image.

The motion data may be incorporated into the forward operator, for example, as described in the above-mentioned publication of L. Cordero-Grande et al. Furthermore, the forward operator may, for example, include a Fourier transform, respective coil sensitivity maps in case of parallel imaging and a projection to the sampled k-space points in case of k-space subsampling.

It is noted that data consistency measure as above are commonly used for iteratively reconstructing MRI images, for example minimizing the data consistency measure under variation of the image space data. Here, however, the data consistency measure is computed based on the already completely reconstructed first preliminary image and used to decide whether the final image shall be reconstructed with or without motion compensation.

For example, the second data consistency measure depends on or is given by ||E x″−y′||, wherein x′ denotes the second preliminary image, E denotes the forward operator without the dependency on the motion data, and y′ denotes the part of the MRI data, which is used for reconstructing the second preliminary image, wherein it may be provided that y′=y.

According to certain embodiments, for reconstructing the first preliminary image, parts of the MRI data are weighted differently depending on respective sampled k-space locations.

For example, k-space regions may be weighted higher the closer they are located to the k-space center. Low-frequency components in k-space generally have higher signal-to-noise ratio, SNR, than peripheral high-frequency components. By assigning greater weight to the k-space center, which is where the most critical image information is located, the reliability of the method is improved leading to more accurate motion correction decisions.

According to certain embodiments, for computing the first data consistency measure, parts of the MRI data are weighted differently depending on respective sampled k-space locations.

The first data consistency measure is then for example given by or depends on ||w(E_{θ}x−y)||, wherein w denotes a respective weighting mask.

According to certain embodiments, only a part of the MRI data for which the motion of the object is neglectable is used for reconstructing the first preliminary image. For example, the MRI data may include a dataset, which may also be denoted as preliminary scan dataset, which corresponds to a preliminary scan of the object, also denoted as scout scan, and the first preliminary image is reconstructed based on the preliminary scan dataset.

This may, for example, be provided as an alternative for compensating the motion of the object when reconstructing the first preliminary image. For example, a scan duration of the scan generating the part of the MRI data which is used for reconstructing the first preliminary image may be very short such that the motion of the object is neglectable. Alternatively or in addition, if the object is a patient, the patient may be instructed not to move at all while the part of the MRI data which is used for reconstructing the first preliminary image is acquired.

The preliminary scan may for example be a SAMER scout scan as described in the 2022 publication of D. Polak et al. mentioned above.

For example, the MRI data includes the preliminary scan dataset, the first preliminary image is reconstructed based on the preliminary scan dataset, and a duration of the preliminary scan is less than ten seconds or less than eight seconds or less than six seconds or less than four seconds. For example, the duration of the preliminary scan may lie in the range [0.5 s, 10 s] or in the range [0.5 s, 8 s] or in the range [0.5 s, 6 s] or in the range [0.5 s, 4 s].

In this way, it is achieved that the motion of the object is neglectable in the first preliminary image.

According to certain embodiments, the MRI data includes one or more first datasets corresponding to one or more first data acquisition shots of the acquisition of the MRI data and one or more second datasets corresponding to one or more second data acquisition shots of the acquisition of the MRI data. The one or more second data acquisition shots are carried out after the one or more first data acquisition shots. The first data consistency measure is computed based on the first preliminary image with respect to the one or more first datasets, for example with respect to only the one or more first datasets. A second data consistency measure is computed based on the first preliminary image with respect to the one or more second datasets, for example with respect to only the one or more second datasets. The final image is reconstructed based on the MRI data, wherein, depending on the first data consistency measure and the second data consistency measure, the motion of the object is either compensated in the final image based on the motion data or is not compensated in the final image.

For example, the preliminary scan may be carried out prior to the one or more first data acquisition shots.

It is noted that each of the one or more first datasets does not necessarily contain all data acquired in the respective first data acquisition shot. In other words, the one or more first datasets correspond to the one or more first data acquisition shots but, in some embodiments, may contain only a part of the data acquired during the one or more first data acquisition shots, respectively.

According to such embodiments, it is decided depending on the first data consistency measure and the second data consistency measure whether the motion of the object is compensated using the motion data when reconstructing the final image or not. For example, the motion of the object is compensated when reconstructing the final image only if it is determined based on the first data consistency measure and the second data consistency measure that a sufficient benefit may be expected due to the motion compensation.

It is noted that in previously described embodiments the first data consistency measure and the second data consistency measure are computed based on the motion-compensated and non-motion-compensated, respectively, in order to assess the benefit of a potential motion compensation of the final image. In the presently described embodiments, on the other hand, the same image, namely the preliminary first image, may for example be used to compute the two different data consistency measures with respect to data acquired at different times in order to assess the benefit of a potential motion compensation of the final image. This is based on the insight that a change in data consistency also may serve as a suitable indicator for the benefit of the motion compensation.

For example, the first data consistency measure depends on or is given by ||E₁x−y₁||, wherein x denotes the first preliminary image, E₁ denotes a predefined first forward operator, and y₁ denotes the one or more first datasets. For example, E₁ depends on further first motion data, which quantifies the motion of the object during the one or more first data acquisition shots.

For example, the second data consistency measure depends on or is given by ||E₂x−y₂||, wherein x denotes the first preliminary image, E₂ denotes a predefined second forward operator, and y₂ denotes the one or more second datasets. For example, E₂ depends on further second motion data, which quantifies the motion of the object during the one or more second data acquisition shots. In other words, the second data consistency measure is computed with motion compensation

The further data consistency measures may be computed analogously.

In some embodiments, the second data consistency measure is computed with motion compensation or, in other words, depending on the respective further second motion data.

In some embodiments, a reference second data consistency measure is computed based on the first preliminary image with respect to the one or more second datasets without compensating for object motion or, in other words, independent of the respective further second motion data or without taking into account the further second motion data. The final image is reconstructed based on the MRI data, wherein, depending on the first data consistency measure, the second data consistency measure and the reference second data consistency measure, the motion of the object is either compensated in the final image based on the motion data or is not compensated in the final image. For example, the final image is reconstructed based on the MRI data, wherein, depending on the first data consistency measure and depending on a difference between the second data consistency measure and the reference second data consistency measure, the motion of the object is either compensated in the final image based on the motion data or is not compensated in the final image.

For example, the second data consistency measure and the reference second data consistency measure may be computed identically except that the respective further second motion is used for computing the second data consistency measure but is not used, for example by setting its values to zero, for computing the reference second data consistency measure.

In some embodiments, it may be provided that the first image is used to compute the two different data consistency measures for detecting parts of the data that should be excluded from an image reconstruction. Such data gaps may be compensated for by the rest of the data, for example if the gaps are not too big.

According to certain embodiments, a data consistency variation measure is computed depending on the first data consistency measure and the second data consistency measure. The final image is reconstructed based on the MRI data, wherein, depending on the data consistency variation measure, the motion of the object is either compensated in the final image based on the motion data or is not compensated in the final image.

In this case, it is decided depending on the data consistency variation measure whether the motion of the object is compensated using the motion data when reconstructing the final image or not. For example, the motion of the object is compensated when reconstructing the final image only if it is determined based on the data consistency variation measure that a sufficient benefit may be expected due to the motion compensation.

For example, it may be provided that the motion of the object is compensated based on the motion data in the final image if the data consistency variation measure is greater than a predefined further threshold value and the motion of the object is not compensated in the final image otherwise.

The data consistency variation measure may for example be or depend on a difference between the first data consistency measure and the second data consistency measure or an absolute value of the difference.

It is, however, also possible that, apart from the one or more first data acquisition shots and the one or more second data acquisition shots, further data acquisition shots or sequences of further data acquisition shots are carried out and a respective further data consistency measure is computed for each further data acquisition shot or sequence of further data acquisition shots at different times. In this case the data consistency variation measure may be computed based on the first data consistency measure, the second data consistency measure, and the further data consistency measures. In this way, a more reliable estimation of the expected benefit of the motion compensation is possible.

In such embodiments, the data consistency variation measure may for example be or depend on a maximum difference of all pairs of the first data consistency measure, the second data consistency measure, and the further data consistency measures, a variance or standard deviation of the first data consistency measure, the second data consistency measure, and the further data consistency measures, et cetera.

Optionally, a respective reference data consistency measure may be computed for each of the further data acquisition shots without motion compensation as described above for the second data consistency measure. For computing the data consistency variation measure, instead of using the second data consistency measure and the further data consistency measures, the difference between the second data consistency measure and the reference second data consistency measure as well as the respective differences between the further data consistency measures and their respective reference further data consistency measures may be used.

The motion data may be determined in a known way as described above. The motion data may also be determined in an iterative approach or optimization approach as described in the 2022 publication of D. Polak et al. mentioned above. Therein, the motion data for a given acquisition shot is varied in multiple optimization steps in order to minimize a respective data consistency error, which is given by the difference between a term given by the preliminary scan dataset to which a forward operator is applied, which depends on the current motion data, from motion guidance data acquired during the respective acquisition shot.

It is noted that each of the one or more first datasets does not necessarily contain all data acquired in the respective first data acquisition shot. For example, the one or more first datasets may correspond to the motion guidance data of the one or more first data acquisition shots and the one or more second datasets may correspond to the motion guidance data of the one or more second data acquisition shots. In other words, the procedure of determining the motion data is exploited additionally to track the data consistency in order to estimate the expected benefit of the motion-compensation in the reconstruction of the final image.

According to certain embodiments, the data consistency variation measure takes into account the change in data consistency, for example data consistency with motion compensation versus data consistency without motion compensation, over time.

According to certain embodiments, a significance class is determined by applying a machine learning model, MLM, for example a trained MLM, to input data, which depends on or includes the first data consistency measure and the second data consistency measure and, for example, the further data consistency measures. The final image is reconstructed based on the MRI data, wherein, depending on the significance class, the motion of the object is either compensated in the final image based on the motion data or is not compensated in the final image.

The MLM is or has been, respectively, trained as a classification model to assign one of two or more significance classes to the data consistency measures. For example, a first data significance class of the two or more significance classes indicates a significant variation of the data consistency and a second significance class of the two or more significance classes indicates an insignificant variation of the data consistency. For example, it may be provided that the motion of the object is compensated based on the motion data in the final image if the first significance class is determined depending on the input data and the motion of the object is not compensated in the final image if the second significance class is determined depending on the input data.

In this way, a particularly reliable prediction of the estimation of the expected benefit of the motion-compensation in the reconstruction of the final image is possible.

In general terms, an MLM, for example a trained MLM, may mimic cognitive functions that humans associate with other human minds. For example, by training based on training data the MLM may be able to adapt to new circumstances and to detect and extrapolate patterns. Another term for a trained MLM is “trained function”.

In general, parameters of an MLM may be adapted or updated by training. For example, supervised training, semi-supervised training, unsupervised training, reinforcement learning and/or active learning may be used. Furthermore, representation learning, also denoted as feature learning, may be used. For example, the parameters of the MLMs may be adapted iteratively by several steps of training. For example, within the training a certain loss function, also denoted as cost function, may be minimized. For example, within the training of an artificial neural network, ANN, the backpropagation algorithm may be used.

For example, an MLM may include an ANN, a support vector machine, a decision tree and/or a Bayesian network, and/or the MLM may be based on k-means clustering, Q-learning, genetic algorithms and/or association rules. For example, an ANN may be or include a fully connected network, a multi-layer perceptron, MLP, a deep neural network, a convolutional neural network or a convolutional deep neural network. Furthermore, an ANN may be an adversarial network, a deep adversarial network and/or a generative adversarial network, GAN.

In some implementations, the MLM is trained in a supervised fashion based on a plurality of training datasets and ground truth data, each training dataset including a respective first data consistency measure, a respective second data consistency measure and, for example, respective further data consistency measures. The ground truth data includes respective annotations or ground truths for each training dataset, the ground truth including a respective ground truth significance class.

The MLM is applied to one of the training datasets to predict a respective predicted significance class. A predefined loss function is evaluated depending on the predicted significance class and the respective ground truth significance class, for example depending on respective deviations of the predicted significance class from the respective ground truth significance class. The MLM is the adapted or updated depending on a result of the evaluation of the loss function. This is repeated for all training datasets or at least for a subset of the plurality of training datasets until a predefined termination criterion or convergence criterion is met.

According to certain embodiments, for each dataset of a plurality of datasets of the MRI data, which correspond to a plurality of subsequent data acquisition shots of the acquisition of the MRI data, a respective data consistency measure is computed based on the first preliminary image with respect to the respective dataset. The final image is reconstructed based on the MRI data, wherein, depending on the data consistency measures computed for the plurality of datasets, the motion of the object is either compensated in the final image based on the motion data or is not compensated in the final image.

For example, the plurality of subsequent data acquisition shots includes the one or more first data acquisition shots, the one or more second data acquisition shots, and the further data acquisition shots. Consequently, the data consistency measures computed for the plurality of datasets include the first data consistency measure, the second data consistency measure, and the further data consistency measures.

For example, a data consistency variation measure is computed, as described above, depending on the data consistency measures of the plurality of datasets. The final image is reconstructed based on the MRI data, wherein, depending on the data consistency measures computed for the plurality of datasets, the motion of the object is either compensated in the final image based on the motion data or is not compensated in the final image.

According to certain embodiments, parts of the MRI data may be excluded from the process of reconstructing the final image based on the MRI data depending on the data consistency measures computed for the plurality of datasets.

For example, if it is found that certain data acquisition shots are affected particularly strongly by motion according to the data consistency measures, the respective k-space date may be ignored or not used, respectively, for reconstructing the final image. Thus, motion artifacts may be suppressed even more effectively.

According to certain embodiments, significance class of the motion of the object is determined by classifying the motion of the object according to at least two significance classes based on the data consistency measures computed for the plurality of datasets. The final image is reconstructed based on the MRI data, wherein, depending on the determined significance class of the motion of the object, the motion of the object is either compensated in the final image based on the motion data or is not compensated in the final image.

The classification may be carried out by an MLM as described above or a by a conventional classification algorithm.

According to a further aspect, a data processing system that is configured to carry out a computer-implemented method is provided.

In the present disclosure, the terms “data processing system” and “at least one data processing device” may be used interchangeably. For example, a data processing device may be understood to mean a data processing device that contains a processing circuit. The data processing device may therefore, for example, process data for the purpose of performing computing operations. This may also include operations for performing indexed access to a data structure, for example a look-up table, LUT, as well as a data processing method implemented in hardware.

The data processing device may include, for example, one or more computers, one or more microcontrollers and/or one or more integrated circuits, for example one or more application-specific integrated circuits, ASIC, one or more field-programmable gate arrays, FPGA, and/or one or more systems-on-a-chip, SoC. The data processing device may also include one or more processors, for example one or more microprocessors, one or more central processing units, CPUs, one or more graphics processing units, GPUs, and/or one or more signal processors, for example one or more digital signal processors, DSPs. The data processing device may also include a physical or virtual network of computers or other of the mentioned units.

In various embodiments, the data processing device includes one or more hardware and/or software interfaces and/or one or more storage units.

A storage unit may be a volatile data memory, for example a dynamic random access memory, DRAM, or a static random access memory, SRAM, or a non-volatile data memory, for example a read-only memory, ROM, a programmable read-only memory, PROM, an erasable programmable read-only memory, EPROM, an electrically erasable programmable read-only memory, EEPROM, a flash memory or flash EEPROM, a ferroelectric random access memory, FRAM, a magnetoresistive random access memory, MRAM, or a phase-change random access memory, PCRAM.

According to a further aspect, an MRI system is provided. The MRI system includes a data processing system and an MRI device, also denoted as MRI scanner. The MRI device is configured to generate the MRI data.

Further implementations of the MRI system follow directly from the various embodiments of the method and vice versa. For example, individual features and corresponding explanations as well as advantages relating to the various implementations of the method may be transferred analogously to corresponding implementations of the MRI system. For example, the MRI system is configured or programmed to carry out the method for image reconstruction. For example, the MRI system carries out the method for image reconstruction.

According to a further aspect, a computer program product including instructions is provided. When the instructions are executed by a data processing system, the instructions cause the data processing system to carry out a computer-implemented method.

The instructions may be provided as program code, for example. The program code may for example be provided as binary code or assembler and/or as source code of a programming language, for example C, and/or as program script, for example Python.

The computer program product may, for example, be a computer program with the instructions or a computer-readable storage medium, for example a tangible and/or non-transient computer readable storage medium, storing a computer program with the instructions.

In the following, embodiments are described in detail with reference to specific implementations and respective schematic drawings. In the drawings, identical or functionally identical elements may be denoted by the same reference signs. The description of identical or functionally identical elements is not necessarily repeated with respect to different figures.

### BRIEF DESCRIPTION OF THE FIGURES

FIG. 1 depicts schematically an embodiment of an MRI system.

FIG. 2 depicts a schematic flow diagram of an embodiment of a computer-implemented method for image reconstruction in MRI.

FIG. 3 depicts a schematic flow diagram of a further embodiment of a computer-implemented method for image reconstruction in MRI.

FIG. 4 depicts a schematic flow diagram of a further embodiment of a computer-implemented method for image reconstruction in MRI.

FIG. 5 depicts a course of data consistency measures during an iterative MRI reconstruction according to an embodiment.

FIG. 6 depicts a course of data consistency measures during a further iterative MRI reconstruction according to an embodiment.

### DETAILED DESCRIPTION

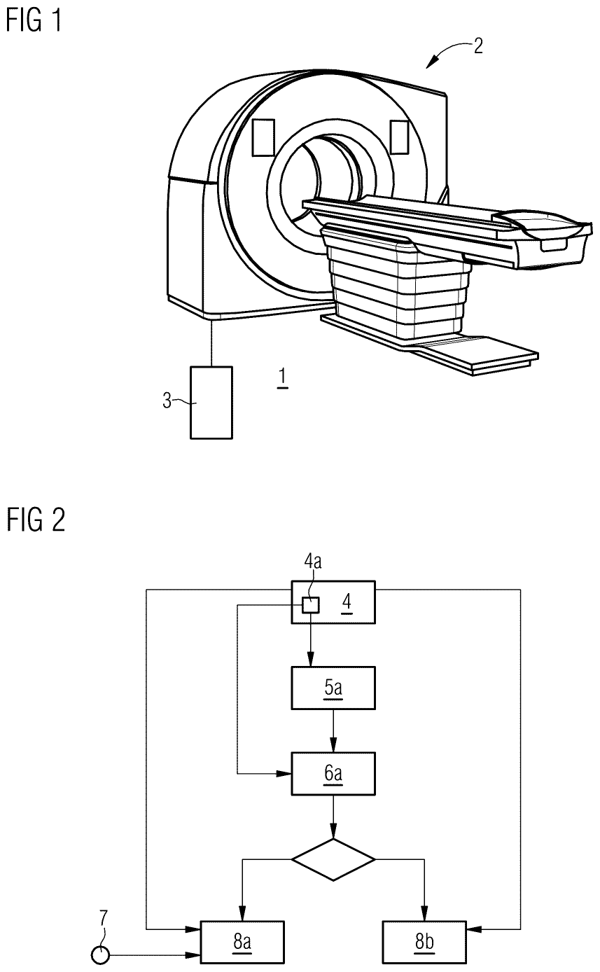

FIG. 1 depicts schematically an embodiment of an MRI system 1. The MRI system 1 includes a data processing system 3 and an MRI device 2, which is configured to generate MRI data 4 (see FIG. 2 to FIG. 4) representing an object. The data processing system 3 is configured to carry out a computer-implemented method for image reconstruction in MRI based on the MRI data 4.
FIG. 2 depicts a schematic flow diagram of an embodiment of a computer-implemented method for image reconstruction in MRI, which may be carried out by the data processing system 3 of FIG. 1.

Therein, a first preliminary image 5a is reconstructed based on the MRI data 4, for example based on a part 4a of the MRI data 4. A first data consistency measure 6a of the first preliminary image 5a with respect to the part 4a of the MRI data 4 is computed. A final image 8a, 8b is reconstructed based on the MRI data 4, for example on the whole MRI data 4 or base on the part 4a and a further part of the MRI data 4. Therein, it is decided depending on the first data consistency measure 6a, whether or not a motion of the object during an acquisition of the MRI data 4 is compensated in the final image 8a, 8b based on predetermined motion data 7, which quantifies the motion of the object during the acquisition of the MRI data 4. If the motion is compensated, a motion-compensated final image 8a results and otherwise a non-motion-compensated final image 8b. In both cases, a predefined image quality, for example a spatial resolution, of the first preliminary image 5a is less than the image quality, for example the spatial resolution, of the final image 8a, 8b.

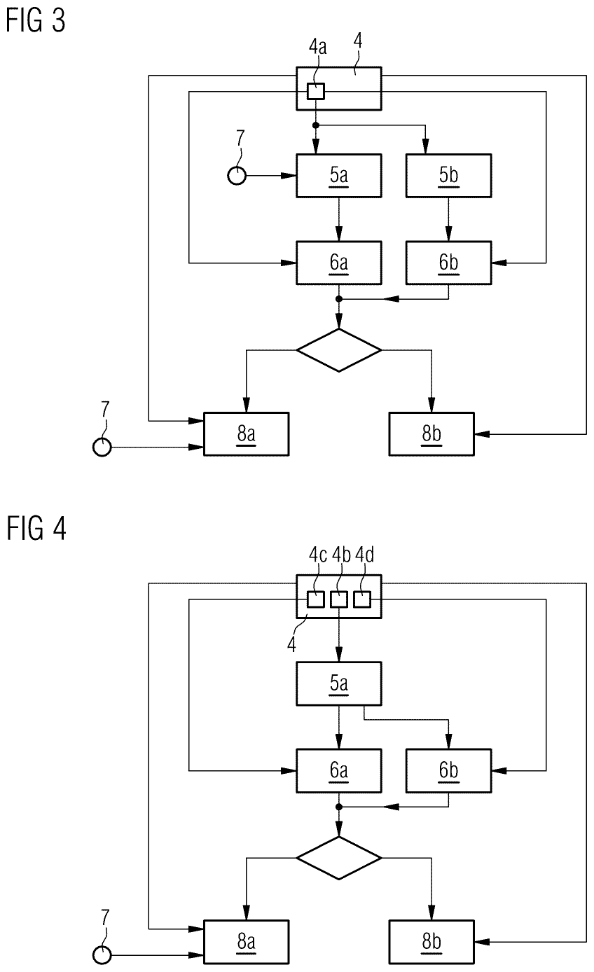

FIG. 3 depicts a schematic flow diagram of a further embodiment of a computer-implemented method for image reconstruction in MRI, which is based on the method of FIG. 2.

In the embodiment of FIG. 3, a second preliminary image 5b is reconstructed based on the MRI data 4, for example based on the part 4a of the MRI data 4. The image quality of the second preliminary image 5b is less than the image quality of the final image 8a, 8b. Therein, the motion of the object is compensated in the first preliminary image 5a based on the motion data 7 and is not compensated in the second preliminary image 5b.

A second data consistency measure 6b of the second preliminary image 5b with respect to the MRI data 4, for example with respect to the part 4a, is computed.

For example, the first data consistency measure 6a may be given by ||E_{θ}x₁−y||, wherein x₁ denotes the first preliminary image 5a, θ denotes the motion data 7, E_{θ} denotes a predefined forward operator, which depends on the motion data 7, and y denotes the part 4a of the MRI data 4, which is used for generating the first preliminary image 5a. For example, the motion data 7 includes three translation parameters and three rotation parameters as a function of time during the acquisition of the MRI data 4.

Analogously, the second data consistency measure 6b may be given by ||E₀ x₂−y||, wherein x₂ denotes the second preliminary image 5b, E₀ is equal to the forward operator E_{θ} except that no motion of the object is considered, or, in other words, the translation parameters and the rotation parameters are set to zero in E₀.

A data consistency deviation between the first data consistency measure 6a and the second data consistency measure 6b is computed, for example as a difference between the first data consistency measure 6a and the second data consistency measure 6b. The final image 8a, 8b with or without the motion compensation depending on the data consistency deviation. For example, the motion-compensated final image 8a is reconstructed if the data consistency deviation is greater than a predefined threshold value and the non-motion-compensated final image 8b is reconstructed otherwise.

Embodiments according to FIG. 3 may, for example, monitor the deviation of the so-called “SENSE+” data consistency errors (see the publication of L. Cordero-Grande et al. mentioned above) between the motion-corrected and uncorrected preliminary images 5a, 5b In general, the motion-corrected first preliminary image 5a is expected to align better with the acquired k-space data 4a than the uncorrected second preliminary image 5b, leading to a reduction in data consistency error in the latter. One significant advantage of this approach is that it eliminates the need for explicit image-based metrics, such as image entropy, which are often challenging to tune and may not consistently yield reliable results. This issue that reconstructions are rather time-consuming is mitigated by conducting the preliminary images 5a, 5b with reduced image quality. Based on the evaluation of the data consistency deviation, the algorithm may then trigger the more computationally intensive reconstruction of the high-resolution final image 8a, 8b and include motion correction only when necessary.

To further improve the reliability of the approach, non-rigid motion effects may be suppressed during reconstruction. In head imaging, for example, the brain may typically be modeled using rigid-body motion in very good approximation, while the neck or mouth region may exhibit non-rigid movement. Techniques such as readout cropping or channel mixing may be used to focus the reconstruction on the desired region, for example the brain, thereby effectively minimizing non-rigid motion effects and improving the accuracy of the data consistency evaluation.

Additionally, it may be beneficial to apply different weighting to regions of k-space. The low-frequency components of k-space generally have higher SNR than the peripheral high-frequency components. By assigning greater weight to the k-space center, where the most critical image information is located, the reliability of the proposed metric may be enhanced, leading to more accurate motion correction decisions.

FIG. 4 depicts a schematic flow diagram of a further embodiment of a computer-implemented method for image reconstruction in MRI, which is based on the method of FIG. 2.

In the embodiment of FIG. 4, only a part of the MRI data 4 for which the motion of the object is neglectable is used for generating the first preliminary image. For example, the MRI data 4 includes a dataset 4b, which corresponds to a preliminary scan of the object, wherein a duration of the preliminary scan is less than ten seconds, for example in the range from two to six seconds, and the first preliminary image 5a is generated based on the dataset 4b.

Furthermore, the MRI data 4 includes one or more first datasets 4c corresponding to one or more first data acquisition shots of the acquisition of the MRI data 4 and one or more second datasets 4d corresponding to one or more second data acquisition shots of the acquisition of the MRI data 4, wherein the one or more second data acquisition shots are carried out after the one or more first data acquisition shots. The first data consistency measure 6a is computed based on the first preliminary image 5a with respect to the one or more first datasets 4c and the second data consistency measure 6b is computed based on the first preliminary image 5a with respect to the one or more second datasets 4d. The final image 8a, 8b is reconstructed with or without the motion compensation based on the motion data 7 depending on the first data consistency measure 6a and the second data consistency measure 6b.

For example, the approach of FIG. 4 may be extended to more than just the two datasets 4c, 4d and the respective data consistency measures 6a, 6b. For example, for each dataset of a plurality of datasets of the MRI data 4 including the datasets 4c, 4d, which correspond to a plurality of subsequent data acquisition shots including the first data acquisition shots and the second data acquisition shots, respectively, a respective data consistency measure 6a, 6b is computed based on the first preliminary image 5a with respect to the respective dataset 4c, 4d. The final image 8a, 8b is then reconstructed based on the MRI data 4 either with or without motion compensation depending on the data consistency measures 6a, 6b computed for the plurality of datasets 4c, 4d, for example depending on a data consistency variation measure, which is determined based on the data consistency measures 6a, 6b. The data consistency variation measure may for example be a maximum difference of all pairs of the data consistency measures, or a variance or standard deviation of the data consistency measures.

In embodiments according to FIG. 4, the motion correction decision may, for example, be guided by tracking the per-shot data consistency errors from the motion estimation. This approach is based on the motion-free or approximately motion-free first preliminary image 5a as a reference, which may, for example, be obtained either after the final iteration of an alternating optimization method or directly from a SAMER scout scan. By passing the image estimate or scout through the motion forward model and comparing it against the acquired k-space data or motion guidance lines from each shot, changes in the data consistency over time may be monitored. For example, small, stable data consistency values suggest minimal or no motion, whereas larger fluctuations may indicate significant motion, prompting a motion-corrected reconstruction.

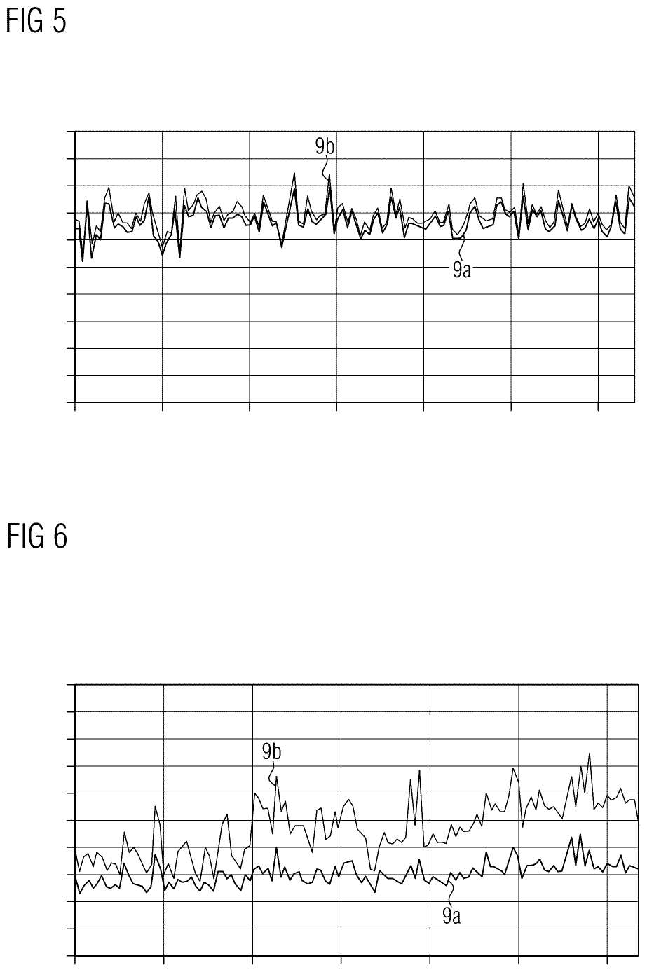

The data consistency measure is shown schematically in FIG. 5 and FIG. 6, wherein a first curve 9a represents the first data consistency measure 5a over many shots, while a second curve 9a represents the second data consistency measure 5b over many shots. In the case of FIG. 5, the difference between the curves 9a, 9b is rather stable. Thus, the final image 8a, 8b may be reconstructed without motion compensation. In the case of FIG. 6, the difference between the curves 9a, 9b increases strongly over time. Thus, the final image 8a, 8b may be reconstructed with motion compensation.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for image reconstruction in magnetic resonance imaging, the method comprising:
receiving MRI data representing an object;
reconstructing a first preliminary image based on the MRI data;
computing a first data consistency measure of the first preliminary image with respect to the MRI data; and
reconstructing a final image based on the MRI data, wherein, depending on the first data consistency measure, a motion of the object during an acquisition of the MRI data is either compensated in the final image based on predetermined motion data that quantifies the motion of the object during the acquisition of the MRI data or is not compensated in the final image;
wherein a predefined image quality of the first preliminary image is less than the image quality of the final image.

2. The computer-implemented method of claim 1, wherein the image quality is a spatial resolution.

3. The computer-implemented method of claim 1, wherein the motion of the object during the acquisition of the MRI data is compensated in the first preliminary image based on the motion data.

4. The computer-implemented method of claim 3, further comprising:
reconstructing a second preliminary image based on the MRI data, wherein the motion of the object is not compensated in the second preliminary image and the image quality of the second preliminary image is less than the image quality of the final image;
computing a second data consistency measure of the second preliminary image with respect to the MRI data; and
computing a data consistency deviation between the first data consistency measure and the second data consistency measure;
wherein the final image reconstructed based on the MRI data wherein, depending on the data consistency deviation, the motion of the object is either compensated based on the motion data in the final image or is not compensated in the final image.

5. The computer-implemented method of claim 4, wherein the first data consistency measure depends on

||E_{θ}x−y||,
wherein x denotes the first preliminary image, θ denotes the motion data, E_{θ} denotes a predefined forward operator, which depends on the motion data, and y denotes a part of the MRI data, which is used for generating the first preliminary image.

6. The computer-implemented method of claim 1, wherein only a part of the MRI data for which the motion of the object is neglectable is used for reconstructing the first preliminary image and/or the MRI data comprises a dataset that corresponds to a preliminary scan of the object, wherein a duration of the preliminary scan is less than ten seconds, and the first preliminary image is reconstructed based on the dataset.

7. The computer-implemented method of claim 6, wherein the MRI data comprises one or more first datasets corresponding to one or more first data acquisition shots of the acquisition of the MRI data and one or more second datasets corresponding to one or more second data acquisition shots of the acquisition of the MRI data, wherein the one or more second data acquisition shots are carried out after the one or more first data acquisition shots;
wherein the first data consistency measure is computed based on the first preliminary image with respect to the one or more first datasets and a second data consistency measure is computed based on the first preliminary image with respect to the one or more second datasets; and
wherein the final image is reconstructed based on the MRI data, wherein, depending on the first data consistency measure and the second data consistency measure, the motion of the object is either compensated in the final image based on the motion data or is not compensated in the final image.

8. The computer-implemented method of claim 7, further comprising:
computing a data consistency variation measure depending on the first data consistency measure and the second data consistency measure, wherein the final image is reconstructed based on the MRI data, wherein, depending on the data consistency variation measure, the motion of the object is either compensated in the final image based on the motion data or is not compensated in the final image.

9. The computer-implemented method of claim 7, further comprising:
determining a significance class by applying a machine learning model to input data, that depends on or comprises the first data consistency measure and the second data consistency measure, wherein the final image is reconstructed based on the MRI data, wherein, depending on the significance class, the motion of the object is either compensated in the final image based on the motion data or is not compensated in the final image.

10. The computer-implemented method of claim 6, further comprising:
computing a respective data consistency measure for each dataset of a plurality of datasets of the MRI data that correspond to a plurality of subsequent data acquisition shots based on the first preliminary image with respect to the respective dataset;
wherein the final image is reconstructed based on the MRI data, wherein, depending on the data consistency measures computed for the plurality of datasets, the motion of the object is either compensated in the final image based on the motion data or is not compensated in the final image.

11. The computer-implemented method of claim 10, further comprising:
determining a significance class of the motion of the object by classifying the motion of the object according to at least two significance classes based on the data consistency measures computed for the plurality of datasets; and
reconstructing the final image based on the MRI data, wherein, depending on the determined significance class of the motion of the object, the motion of the object is either compensated in the final image based on the motion data or is not compensated in the final image.

12. The computer-implemented method of claim 1, wherein, for reconstructing the first preliminary image and/or for computing the first data consistency measure, parts of the MRI data are weighted differently depending on respective sampled k-space locations.

13. The computer-implemented method of claim 1, wherein the motion data comprises one, two or three translation parameters as a function of time during the acquisition of the MRI data and/or one, two, or three rotation parameters as a function of time during the acquisition of the MRI data or a motion field as a function of time during the acquisition of the MRI data.

14. A MRI system comprising:
an MRI device configured to generate MRI data of an object; and
a data processing system configured to:
receive the MRI data;
reconstruct a first preliminary image based on the MRI data;
compute a first data consistency measure of the first preliminary image with respect to the MRI data; and
reconstruct a final image based on the MRI data, wherein, depending on the first data consistency measure, a motion of the object during an acquisition of the MRI data is either compensated in the final image based on predetermined motion data that quantifies the motion of the object during the acquisition of the MRI data or is not compensated in the final image;
wherein a predefined image quality of the first preliminary image is less than the image quality of the final image.

15. A non-transitory computer implemented storage medium that stores machine-readable instructions executable by at least one processor, the machine-readable instructions comprising:
receiving MRI data representing an object;
reconstructing a first preliminary image based on the MRI data;
computing a first data consistency measure of the first preliminary image with respect to the MRI data; and
reconstructing a final image based on the MRI data, wherein, depending on the first data consistency measure, a motion of the object during an acquisition of the MRI data is either compensated in the final image based on predetermined motion data that quantifies the motion of the object during the acquisition of the MRI data or is not compensated in the final image;
wherein a predefined image quality of the first preliminary image is less than the image quality of the final image.
